(19) United States
(12) Patent Application Publication
(10) Pub. No.: US 2016/0201138 A1
(43) Pub. Date: Jul. 14, 2016

(54) METHOD AND MARKERS FOR ASSESSING THE RISK OF HAVING COLORECTAL CANCER

(71) Applicants: Chang Gung University, Taoyuan City (TW); Chang Gung Memorial Hospital, Linkou, Taoyuan City (TW)

(72) Inventors: Jinn- Shiun CHEN, Taipei City (TW); Yu-Sun CHANG, New Taipei City (TW); Err-Cheng CHAN, Taipei City (TW); Chia-Chun CHEN, Taipei City (TW); Pi-Yueh CHANG, New Taipei City (TW)

(21) Appl. No.: 14/928,387

(22) Filed: Oct. 30, 2015

(30) Foreign Application Priority Data
Jan. 9, 2015 (TW) 104100830

(51) Int. Cl. C12Q 1/68

(52) U.S. Cl. CPC C12Q 1/6886; C12Q 2600/158; C12Q 2600/178; C12Q 2600/118

(57) ABSTRACT

The disclosure discloses a method and markers for assessing the risk of having colorectal cancer for an individual by a stool sample. The method includes: detecting expression levels of a first microRNA and a second microRNA in the stool sample; and assessing the risk of having colorectal cancer for the individual based on a ratio between the expression levels of the first microRNA and the second microRNA. Here, the first microRNA is miR-223, miR-25, or miR-93, and the second microRNA is miR-221, miR-222, miR-21, miR-93, miR-141, miR-200c, miR-191, miR-17, miR-148a, miR-106a, miR-195, miR-20a, miR-181b, miR-145, miR-155, miR-106b, miR-24, miR-19b, miR-130b, or miR-18a. When the first microRNA is miR-93, the second microRNA is miR-17, miR-106a, miR-195, miR-20a, miR-181b, miR-155, miR-24, miR-19b, or miR-18a.

### CROSS REFERENCE TO RELATED APPLICATIONS

This Non-provisional application claims priority under 35 U.S.C. §119(a) on Patent Application No(s). 104100830 filed in Taiwan, Republic of China on Jan. 9, 2015, the entire contents of which are hereby incorporated by reference.

### BACKGROUND OF THE INVENTION

1. Field of Invention

The invention relates to a method and markers for assessing the risk of having colorectal cancer for an individual.

2. Related Art

Micro-ribonucleic acids are also known as miRNAs, mi-RNAs, and microRNAs. MicroRNAs regulate gene expression in an organism mainly through degradation of messenger ribonucleic acid (mRNA) or inhibition of translational mechanism. They are important in regulating growth and development of animals and plants, differentiation and apoptosis of cells, and human diseases (tumors for example). Moreover, the special function of microRNA is closely related to the pathogenesis of tumor, so it is highly valued in tumor classification and prediction. The detection of miRNA contributes to precise tumor typing and grasp of tumor heterogeneity, and medication can become more accurate and effective.

Colorectal cancer (CRC) is one of the top 4 deadly cancer. About 700,000 people died of this cancer around the world every year. Early-stage cancer (before metastasis) may possibly be cured through surgery. However, symptoms of colorectal cancer, for example bloody stools or changes in bowel habits, are often ignored because they are unobvious, so patients are often diagnosed to have colorectal cancer at the late stage of cancer (after the cancer is metastasized). Therefore, early diagnosis of colorectal cancer is quite important. If colorectal cancer is detected early, the prognosis is much better than the prognosis of colorectal cancer detected at late stage at which cancer has already spread.

Currently, the conventional method for assessing the risk of having colorectal cancer is an examination by a variety of endoscopes or tomography instruments, a fecal occult blood test (FOBT), or the like. However, the result of the tomography is often inaccurate due to its image resolution. Endoscopy is risky because it is an invasive examination. Although the fecal occult blood test has advantages of low cost and simple operation, its accuracy is not high. Currently, an immunochemical fecal occult blood test can avoid false negative and false positive errors caused by the diet of patients, but its accuracy still needs to be improved. Therefore, conventional methods for assessing the risk of having colorectal cancer have low accurate assessment results or they are invasive detections. Therefore, another method for assessing the risk of having colorectal cancer is needed for non-invasive detection with high sensitivity.

### SUMMARY OF THE INVENTION

An aspect of the disclosure is to provide a method and markers for assessing the risk of having colorectal cancer for an individual by a stool sample of the individual so as to perform a non-invasive detection with high sensitivity. Here, the markers are specific combinations of microRNAs in the stool sample. The expression levels of the specific combinations of microRNAs are detected to assess the risk of having colorectal cancer for the individual.

A method for accessing the risk of having colorectal cancer for an individual by a stool sample obtained from the individual is provided. The method includes the steps of: detecting expression levels of a first microRNA (miRNA) and a second microRNA in the stool sample, and assessing the risk of having colorectal cancer for the individual based on a ratio between the expression levels of the first microRNA and the second microRNA.

The term “microRNA” recited in the specification means a small ribonucleic acid which can be synthesized in an organism (an individual is for example in embodiments) and contains about 22 nucleotides. MicroRNAs are non-coding RNAs, that is to say, microRNAs will not be translated into corresponding proteins. However, microRNAs still function in regulation of gene expression, such as regulation of cell growth, cell differentiation, apoptosis, cancer formation, and the like in an organism.

Moreover, a microRNA generally regulates gene expression by binding to complementary sequences within a messenger RNA (also known as mRNA) so as to result in degradation of the mRNA or inhibition of translation. In addition, microRNA research also points out that microRNAs are related to the pathological mechanism of human cancer, for example, a specific microRNA can regulate gene expression related to a specific cancer. Accordingly, the expression levels of the corresponding microRNAs are different in different cancer patients. In this embodiment, characteristics of microRNA are used to develop a method for assessing the risk of having colorectal cancer for an individual. Namely expression levels of specific microRNAs in the individual are detected so as to assess the risk of having colorectal cancer for the individual based on the expression levels. Its embodiments are illustrated in the below description.

The term “expression level of microRNA” recited in the specification means the content of the microRNA in an individual, and it refers to the content of the microRNA in the “stool sample” in this embodiment.

A marker is applied for assessing the risk of having colorectal cancer for an individual by a stool sample obtained from the individual. The marker includes a first microRNA and a second microRNA. The difference between a ratio between expression levels of the first microRNA and the second microRNA in at least one stool sample obtained from a colorectal cancer patient and that in a control stool sample is statistically significant.

The term “marker” recited in the specification means a biomarker for assessing the risk of having colorectal cancer for an individual. Moreover, it refers to the specific combination of microRNAs in the specification, namely the combination of the first microRNA and the second microRNA. In detail, the first microRNA is selected from the group consisting of miR-223, miR-25, and miR-93, and the second microRNA is selected from the group consisting of miR-221, miR-222, miR-21, miR-93, miR-141, miR-200c, miR-191, miR-17, miR-148a, miR-106a, miR-195, miR-20a, miR-181b, miR-145, miR-155, miR-106b, miR-24, miR-19b, miR-130b, and miR-18a. Moreover, when the first microRNA is miR-93, the second microRNA is miR-17, miR-106a, miR-195, miR-20a, miR-181b, miR-155, miR-24, miR-19b, or miR-18a.

In one embodiment, the first microRNA is miR-223, and the second microRNA is miR-221, miR-222, miR-21, or miR-93.

In one embodiment, the first microRNA is miR-25, and the second microRNA is miR-221, miR-222, miR-21, miR-93, miR-141, miR-200c, or miR-191.

In one embodiment, the result of the step of assessing the risk of having colorectal cancer for the individual is high risk if the ratio between the expression levels of the first microRNA and the second microRNA is greater than a detection threshold.

In one embodiment, the step of detecting expression levels of a first microRNA and a second microRNA in the stool sample is followed by the steps: if the expression level of the first microRNA is greater than a concentration threshold, the result of assessing the risk of having colorectal cancer for the individual is high risk, and if the expression level of the first microRNA is less than the concentration threshold, the result of assessing the risk of having colorectal cancer for the individual should be further based on the ratio between the expression levels of the first microRNA and the second microRNA.

Types of first microRNAs and second microRNAs can refer to the above description. Moreover, the term “concentration threshold” recited in the specification means reference values for assessing the expression levels of the first microRNAs or the second microRNAs by the stool sample of the assessment object. In detail, the content of the first microRNA or the second microRNA in the stool sample is measured, and if the concentration is greater than a reference value, namely a concentration threshold called in the invention, the result of the assessment is high risk. This assessment method is established by the difference between the expression levels of microRNAs in colorectal cancer patients and those in healthy individuals.

In one embodiment, the expression level of the first microRNA is up-regulated.

The term “up-regulated” recited in the specification means that the expression level of the first microRNA in the stool sample of the colorectal cancer patient is greater than that of the normal individual.

The invention further provides another method for assessing the risk of having colorectal cancer for an individual by a stool sample obtained from the individual. The method includes the steps of: performing a fecal occult blood test on the stool sample and detecting expression levels of miR-93, miR-155, miR-223, miR-221, and miR-222; selecting the stool sample of positive fecal occult blood test result and assessing the risk of having colorectal cancer for the individual based on a first ratio between the expression levels of miR-93 and miR-155, a second ratio between the expression levels of miR-223 and miR-221, or a third ratio between the expression levels of miR-223 and miR-222, and if anyone of the first ratio, the second ratio, or the third ratio is greater than a corresponding detection threshold, the result of the assessment is high risk; and selecting the stool sample of negative fecal occult blood test result and assessing the risk of having colorectal cancer for the individual based on the first ratio between the expression levels of miR-93 and miR-155, the second ratio between the expression levels of miR-223 and miR-221, and the third ratio between the expression levels of miR-223 and miR-222, and if the first ratio, the second ratio, and the third ratio are all greater than the corresponding detection thresholds, the result of the assessment is high risk.

As mentioned above, the assessment method and the markers are used to assess the risk of having colorectal cancer for an individual based on the ratio between the expression levels of the first microRNA and the second microRNA. Thus, they provide the assessment result of the non-invasive detection with high sensitivity and accuracy.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a flow chart of an assessment method according to an embodiment;

FIG. 2 is a flow chart of an assessment method according to another embodiment;

FIG. 3 is a flow chart of an assessment method according to another embodiment; and

FIG. 4 is a diagram showing the experimental result of each assessment method of Experimental example 3.

### DETAILED DESCRIPTION OF THE INVENTION

The embodiments and experimental examples of the present invention will be apparent from the following detailed description, which proceeds with reference to the accompanying drawings, wherein the same references relate to the same elements. However, the related interpretations of the terms can refer to the above description, so they are not repeated here.

The disclosure provides a method for assessing the risk of having colorectal cancer for an individual by a stool sample obtained from the individual. In the embodiment, this method is called the assessment method, and the individual who is assessed is called the assessment object. The assessment method of the disclosure is to assess the risk of having colorectal cancer by detecting the stool sample of the assessment object.

In the embodiment, the stool sample of the assessment object is collected, dipped with a designated swab (or a stool collection device) and then the swab is inserted into a preservation buffer. The stool sample attached to the swab or the stool collection device is then evenly dissolved in the preservation buffer by thorough vortex, and the preservation buffer is squeezed out into an eppendorf for storage. Reagents for collection used here can be the same collection reagents used in fecal occult blood test (FOBT), for example, OC-Sensor Diana Latex Reagent (Eiken Chemical, Tokyo, Japan), or any other brand of reagents, and the invention is not limited thereto.

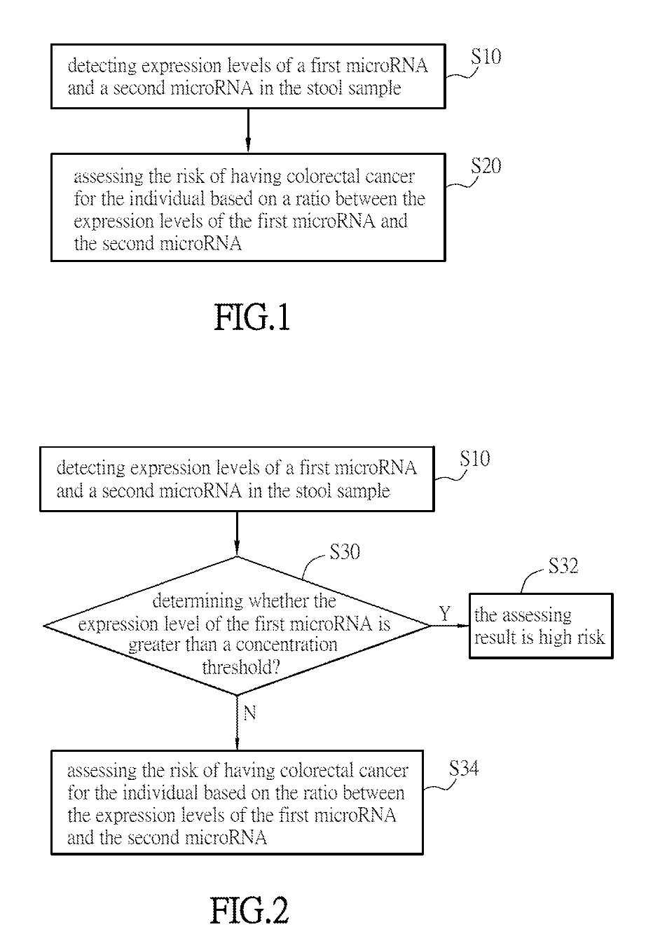

Referring to FIG. 1, it is a flow chart of the assessment method according to the first embodiment. The assessment method of the embodiment includes the steps of: detecting expression levels of a first microRNA and a second microRNA in the stool sample (step S10); and assessing the risk of having colorectal cancer for the individual based on a ratio between the expression levels of the first microRNA and the second microRNA (step S20).

Here, the first microRNA and the second microRNA can respectively be groups consisting of multiple microRNAs. For example, the first microRNA is selected from the group consisting of miR-223, miR-25, and miR-93, and the second microRNA is selected from the group consisting of miR-221, miR-222, miR-21, miR-93, miR-141, miR-200c, miR-191, miR-17, miR-148a, miR-106a, miR-195, miR-20a, miR-181b, miR-145, miR-155, miR-106b, miR-24, miR-19b, miR-130b, and miR-18a. They are listed in Table 1.

TABLE 1
Lists of the first microRNA group and the second microRNA group

| First microRNA group | Second microRNA group |
|---|---|
| miR-223, miR-25, | miR-221, miR-222, miR-21, miR-93, miR-141, |
| and miR-93 | miR-200c, miR-191, miR-17, miR-148a, |
| | miR-106a, miR-195, miR-20a, miR-181b, |
| | miR-145, miR-155, miR-106b, miR-24, miR-19b, |
| | miR-130b, and miR-18a |

It should be noted that when the first microRNA is miR-93, the second microRNA is limited to miR-17, miR-106a, miR-195, miR-20a, miR-181b, miR-155, miR-24, miR-19b, or miR-18a. Therefore, the assessment method described in the embodiment does not include that the first microRNA and the second microRNA are miR-93 simultaneously.

In the step S10, the expression levels of the above mentioned first microRNA and second microRNA in the stool sample are detected. Preferably, the expression levels of the first microRNA and the second microRNA can be detected by a microarray or a quantitative polymerase chain reaction (qPCR) technique. For the microarray, one microarray can be divided into two regions which are respectively provided with the nucleotide probes corresponding to the first microRNA group and the second microRNA group listed in the above table. Alternatively, one microarray is provided with the nucleotide probes corresponding to the first microRNA group listed in the above table, the other microarray is provided with the nucleotide probes corresponding to the second microRNA group listed in the above table, and the detection is performed with two microarrays. For the quantitative polymerase chain reaction, the primers and the nucleotide probes can be designed to detect above mentioned respective first microRNAs and second microRNAs, and the expression levels of respective first microRNAs and second microRNAs are detected by the quantitative polymerase chain reaction.

Moreover, the sequence of each microRNA included in the first microRNA group and the second microRNA group can be found in the disclosed sequences of microRNAs in the online database of miRBase. The corresponding primers and nucleotide probes may be designed according to those sequences, or they may be purchased by entering the corresponding Accession No. on the website of Applied Biosystems as described in Experimental example 1 below.

In the embodiment, the below example illustrates that the expression levels of the first microRNA and the second microRNA are the concentrations of nucleic acid fragments (copies/μl) converted from Cq values obtained by the quantitative polymerase chain reaction. The Cq value (quantification cycle, also known as threshold cycle) refers to a corresponding cycle number if the generation amount of the nucleic acid fragment is greater than a threshold value during the quantitative polymerase chain reaction. Generally, the logarithms of the initial concentrations of the nucleic acid fragment have a linear relationship with the Cq values of the nucleic acid fragment in the quantitative polymerase chain reaction. Therefore, the concentration of the nucleic acid fragment to be measured in an unknown sample can be calculated by comparing the obtained Cq value of the unknown sample with the copy number-Cq value standard curve established by standard samples. Accordingly, values of Cq_{X} and Cq_{Y} are obtained by performing the quantitative polymerase chain reaction on samples of miRNA_{X} and miRNA_{Y} which are two nucleic acid fragments to be measured. The ratio between the initial concentrations of these two nucleic acid fragments to be measured can be calculated by exponentiation using 2 as the base and the difference between the values of Cq_{X} and Cq_{Y} as the exponent. The conversion equation is:

miR_{X}/miR_{Y}=2^{Cq}^{_{Y}}^{-Cq}^{_{X}} (1)

Here, miR_{X} indicates the initial concentration of miRNA_{X}, miR_{Y} indicates the initial concentration of miRNA_{Y}, Cq_{X} is the Cq value of miRNA_{X} obtained by the quantitative polymerase chain reaction, and Cq_{Y} is the Cq value of miRNA_{Y} obtained by the quantitative polymerase chain reaction.

Moreover, a two-step quantitative polymerase chain reaction is illustrated for example in the embodiment. That is to say, total RNA is reversely transcribed into complementary deoxyribonucleic acids (cDNAs), and then the cDNAs act as templates to perform the quantitative polymerase chain reaction. For example, the high speed centrifugation is performed on the stool sample obtained from the assessment object and dissolved in the preservation reagent and, and the supernatant is taken for extraction of total RNA. Then, the reverse transcription polymerase chain reaction is performed on the extracted total RNA with the mixture of primers corresponding to the above described first microRNA group and second microRNA group to obtain cDNAs. Further, the cDNAs act as templates to perform the quantitative polymerase chain reaction with the primers corresponding to the first microRNA group and the second microRNA group respectively in order to obtain Cq values of each first microRNA and each second microRNA described above. The Cq values are converted into ratios between expression levels in the embodiment according to the above equation.

Generally, a signal having over 40 cycles (Cq>40) is considered low reliable in the detection of the quantitative polymerase chain reaction. Thus, in the embodiment, the cycle number of the quantitative polymerase chain reaction is set to 40 according to TaqMan® MicroRNA Assays Protocol. Therefore, the maximum of the Cq value is 40.

After detecting the expression levels of each first microRNA and each second microRNA by the quantitative polymerase chain reaction, in the step S20, the risk of having colorectal cancer is assessed for an individual based on the ratio between the expression levels of the first microRNA and the second microRNA. If the ratio between the expression levels of the first microRNA and the second microRNA is greater than a detection threshold, the assessing result is high risk. It should be noted that the ratio between the expression levels of the first microRNA and the second microRNA in the embodiment may be a specific value obtained by dividing the expression level of the first microRNA by the expression level of the second microRNA (hereinafter referred to as “first microRNA/second microRNA”), for example the specific value of miR-223/miR-221, or a specific value obtained by dividing the expression level of the second microRNA by the expression level of the first microRNA (hereinafter referred to as “second microRNA/first microRNA”), for example the specific value of miR-221/miR-223, and it is not limited thereto. The ranges of the detection thresholds corresponding to different combinations of the first microRNA and the second microRNA and that the applicable combinations are first microRNA/second microRNA or second microRNA/first microRNA are recited in Table 2. Therefore, the risk level for the assessment object having colorectal cancer can be assessed based on Table 2. The term “detection threshold” recited in the specification means a reference value for assessing the risk of having colorectal cancer for an individual. The detection threshold is set within a preferred range of values. In other words, it is not a constant value. The sensitivity and the specificity of the detection changes with the detection threshold. Generally, the detection thresholds which respectively correspond to different combinations of the first microRNA and the second microRNA will be different. The following description will illustrate the detection thresholds suitable for various combinations of the first microRNA and the second microRNA and illustrate the ranges thereof.

As shown in Table 2, in the embodiment, if the ratio between the expression levels of the first microRNA and the second microRNA (i.e. the specific value of first microRNA/second microRNA or second microRNA/first microRNA mentioned above) is greater than the detection threshold, the assessing result is high risk, otherwise the assessing result is low risk. Alternatively, the assessing result is high risk if the ratio between the expression levels of the first microRNA and the second microRNA (i.e. the specific value of first microRNA/second microRNA or second microRNA/first microRNA mentioned above) is greater than the maximum of the range of the detection threshold, the assessing result is medium risk if the ratio is within the range of the detection threshold, or the assessing result is low risk if the ratio is less than the minimum of the range of the detection threshold.

TABLE 2
List of ranges of detection thresholds corresponding to ratios between
expression levels of first microRNAs and the second microRNAs

| | Detection threshold (range) | |
|---|---|---|
| First microRNA/second microRNA | | |
| miR-223/miR-221 | 9.563 | (4.143-13.92) |
| miR-223/miR-222 | 8.846 | (4.798-12.54) |
| miR-223/miR-21 | 0.6263 | (0.317-0.787) |
| miR-223/miR-93 | 1.239 | (0.676-1.897) |
| miR-223/miR-141 | 20.33 | (12.66-28.45) |
| miR-223/miR-200c | 3.706 | (1.697-5.152) |
| miR-223/miR-191 | 42.41 | (25.11-58.04) |
| miR-223/miR-17 | 2.838 | (1.509-3.541) |
| miR-223/miR-148a | 137.5 | (64.19-205.4) |
| miR-223/miR-106a | 9.550 | (5.79-13.95) |
| miR-223/miR-195 | 46.19 | (29.43-67.23) |
| miR-223/miR-20a | 9.458 | (5.79-12.94) |
| miR-223/miR-181b | 61.39 | (24.5-89.83) |
| miR-223/miR-145 | 96.44 | (34.58-168.9) |
| miR-223/miR-155 | 128.4 | (63.18-200) |
| miR-223/miR-106b | 4.233 | (1.689-5.794) |
| miR-223/miR-24 | 47.21 | (23.65-63.29) |
| miR-223/miR-19b | 41.34 | (19.41-56.91) |
| miR-223/miR-130b | 13.93 | (4.649-26.92) |
| miR-223/miR-18a | 42.98 | (19.5-66.67) |
| miR-25/miR-221 | 0.4366 | (0.21-0.666) |
| miR-25/miR-222 | 0.2664 | (0.139-0.386) |
| miR-25/miR-21 | 0.05433 | (0.027-0.076) |
| miR-25/miR-93 | 0.05303 | (0.017-0.092) |
| miR-25/miR-141 | 1.061 | (0.714-1.897) |
| miR-25/miR-200c | 0.2325 | (0.132-0.356) |
| miR-25/miR-191 | 1.118 | (0.965-2.577) |
| miR-25/miR-17 | 0.2446 | (0.125-0.363) |
| miR-25/miR-148a | 8.776 | (3.382-14.08) |
| miR-25/miR-106a | 0.1484 | (0.061-0.202) |
| miR-25/miR-195 | 0.4772 | (0.292-0.724) |
| miR-25/miR-20a | 0.2475 | (0.125-0.36) |
| miR-25/miR-181b | 1.766 | (0.998-3.883) |
| miR-25/miR-145 | 1.176 | (0.972-2.98) |
| miR-25/miR-155 | 1.557 | (0.995-3.246) |
| miR-25/miR-106b | 0.1166 | (0.052-0.185) |
| miR-25/miR-24 | 1.143 | (0.996-2.376) |
| miR-25/miR-19b | 1.105 | (0.993-1.873) |
| miR-25/miR-130b | 1.008 | (0.996-1.988) |
| miR-25/miR-18a | 1.041 | (1-1.96) |
| Second microRNA/first microRNA | | |
| miR-17/miR-93 | 0.1135 | (0.084-0.151) |
| miR-106a/miR-93 | 0.1522 | (0.104-0.191) |
| miR-195 miR-93 | 0.1517 | (0.089-0.209) |
| miR-20a/miR-93 | 0.2473 | (0.172-0.289) |
| miR-181b/miR-93 | 0.01116 | (0.007-0.014) |
| miR-155/miR-93 | 0.005128 | (0.004-0.006) |
| miR-24/miR-93 | 0.1271 | (0.038-0.187) |
| miR-19b/miR-93 | 0.04282 | (0.012-0.06) |
| miR-18a/miR-93 | 0.03339 | (0.018-0.043) |

For example, after the quantitative polymerase chain reaction, a specific value can be obtained by dividing the concentration of miR-223 (first microRNA) by the concentration of miR-221 (second microRNA), or a specific value is calculated with the above equation (1) using the Cq values of miR-223 and miR-221 obtained by the quantitative polymerase chain reaction, and then the specific value is compared with Table 2. The assessing result is high risk of having colorectal cancer if the specific value is greater than 9.563, and it is low risk if the specific value is less than 9.563. As to the assessment with the range of the detection threshold, the assessing result is high risk of having colorectal cancer if the specific value is greater than 13.92, or it is medium risk if the specific value is within 4.143-13.92 (including the specific value is equal to 4.143 or 13.92), or it is low risk if the specific value is less than 4.143.

In the above embodiment, although the risk is assessed high if the ratio between the expression levels of the first microRNA and the second microRNA (i.e. the specific value of first microRNA/second microRNA or second microRNA/first microRNA as mentioned above) is greater than the corresponding detection threshold listed in Table 2 for example, the risk can be assessed by other simple possible variations. For example, a specific value may be obtained by interchanging the numerator and the denominator and it becomes the reciprocal of the original specific value. If the reciprocal specific value is used for assessment, the corresponding detection threshold to be used can be obtained by calculating the reciprocal of the original detection threshold, and the assessment method is changed to that the assessing result is high risk if the specific value is less than the corresponding detection threshold. But, the detection results (area under the ROC curve, sensitivity, and specificity) does not change accordingly.

For example, on condition that the specific value of miR-223/miR-221 as shown in Table 2 is used for assessment, the assessing result is high risk of having colorectal cancer if the specific value of miR-223/miR-221 is greater than 13.92, or it is medium risk if the specific value is within 4.143-13.92 (including the specific value is equal to 4.143 or 13.92), or it is low risk if the specific value is less than 4.143. On condition that the specific value of miR-221/miR-223 is used for assessment, the corresponding range of the detection threshold is 0.072 (the round reciprocal of 13.92) to 0.241 (the round reciprocal of 4.143) after conversion. The assessing result is high risk of having colorectal cancer if the specific value of miR-221/miR-223 is less than 0.072, or it is medium risk if the specific value is within 0.072-0.241 (including the specific value is equal to 0.072 or 0.241), or it is low risk if the specific value is greater than 0.241. Similarly, on condition that the specific value of miR-25/miR-24 as shown in Table 2 is used for assessment, the assessing result is high risk of having colorectal cancer if the specific value of miR-25/miR-24 is greater than 2.376, or it is medium risk if the specific value is within 0.996-2.376 (including the specific value is equal to 0.996 or 2.376), or it is low risk if the specific value is less than 0.996. On condition that the specific value of miR-24/miR-25 is used for assessment, the corresponding range of the detection threshold is 0.421 (the round reciprocal of 2.376) to 1.005 (the round reciprocal of 0.996) after conversion. The assessing result is high risk of having colorectal cancer if the specific value of miR-24/miR-25 is less than 0.421, or it is medium risk if the specific value is within 0.421-1.005 (including the specific value is equal to 0.421 or 1.005), or it is low risk if the specific value is greater than 1.005.

The assessment method of this embodiment includes the microRNA types of the first microRNA group and the second microRNA group shown in Table 1 and the ranges of the detection thresholds shown in Table 2. Stool samples from 144 patients with diagnosed colorectal cancer and from 390 healthy individuals are respectively collected. The contents of microRNAs in the stool samples are calculated by the inventors so as to induce the microRNA types of the first microRNA group and the second microRNA group shown in the above table and obtain the corresponding ranges of the detection thresholds. The detection results (sensitivity and specificity) are shown in Experimental example 2 below.

Because the content of total RNA in a stool sample is extremely low, absolutely quantifying template concentrations is not accurate. In conventional methods for detecting microRNA, the microRNA concentration needs to be amplified first by a polymerase chain reaction (PCR), and then a quantification test is performed. However, the existing problem is that the concentrations of nucleic acid fragments in the stool samples, namely the concentrations of templates for a polymerase chain reaction, may have huge errors due to different collection time, experimental operation, sampling, and other factors. Therefore, it is difficult to control every batch of stool samples to be at the same standard. Accordingly, assessment methods established by merely using detection methods related to the polymerase chain reaction (PCR) have considerable errors.

Therefore, the assessment method according to the first embodiment is to calculate the ratio between the expression levels of the first microRNA and the second microRNA. Differences caused by different volumes of templates can be excluded by dividing the expression levels of the first microRNAs and those of the second microRNAs. As a result, the established assessment method can reduce detection errors caused by the differences between every collection of stool sample.

In one preferable example according to the assessment method shown in the first embodiment, miR-223 is selected from the first microRNA group as a detection target, miR-221, miR-222, miR-21, or miR-93 is selected from the second microRNA group as a detection target, and the ratio between the expression levels of the first microRNA and the second microRNA is compared, namely the specific value of miR-223/miR-221, miR-223/miR-222, miR-223/miR-21, or miR-223/miR-93 is compared. In the embodiment, the area under curve (AUC) by using the ratio between the expression levels of the first microRNA and the second microRNA is greater (i.e., more accurate) than by using only the first microRNA or by using only the second microRNA.

Moreover, the embodiment also provides other possible combination examples listed below. miR-25 is selected from the first microRNA group as a detection target, miR-221, miR-222, miR-21, miR-93, miR-141, miR-200c, or miR-191 is selected from the second microRNA group as a detection target, and the specific value of miR-25/miR-221, miR-25/miR-222, miR-25/miR-21, miR-25/miR-93, miR-25/miR-141, miR-25/miR-200c, or miR-25/miR-191 is compared. Similarly, the area under curve (AUC) is greater (i.e., the result is more accurate) by using the ratio between the expression levels of the first microRNA and the second microRNA than by using only the first microRNA or using only the second microRNA.

Referring to FIG. 2, it is a flow chart of the assessment method according to the second embodiment. The assessment method of this embodiment includes the steps of: detecting expression levels of a first microRNA and a second microRNA in the stool sample (step S10); and determining whether the expression level of the first microRNA is greater than a concentration threshold (step S30). Then, if the result of the step S30 is “yes”, namely when the expression level of the first microRNA is greater than a concentration threshold, the assessing result is high risk (step S32). If the result of the step S30 is “no”, namely when the expression level of the first microRNA is less than the concentration threshold, the result of assessing the risk of having colorectal cancer for the individual should be further based on the ratio between the expression levels of the first microRNA and the second microRNA (step S34). Here, the first microRNA and the second microRNA both are groups consisting of multiple microRNAs, and the details can refer to the first microRNA group and the second microRNA group listed in Table 1.

In the embodiment, the expression levels of the microRNAs are also detected first (step S10), and then whether the expression level of the first microRNA is greater than a concentration threshold is determined (step S30). In detail, if the expression level of the first microRNA (miR-223, miR-25, or miR-93) is greater than a concentration threshold, the assessing result is high risk (step S32). The term “concentration threshold” recited in this embodiment means a reference value for a concentration of nucleic acid fragment (copies/μl). Here, the corresponding predetermined values for the first microRNAs miR-223, miR-25, and miR-93, are shown in Table 3. It should be noted that the reference values for assessment listed in Table 3 are illustrated by taking the preferred concentration thresholds (e.g. 550.6 copies/μl) for example. Certainly, suitable concentration thresholds can also be selected from the ranges of the concentration thresholds (e.g. 226.4-804.8 copies/μl) as reference values for assessment, and the invention is not limited thereto. In other embodiments, if other first microRNAs are used, the corresponding concentration thresholds become different. The step S30 assesses the risk level of having colorectal cancer for an individual depending on whether the expression level of the first microRNA is greater than the corresponding concentration threshold.

TABLE 3
Concentration thresholds and preferred ranges thereof
for first microRNAs miR-223, miR-25 and miR-93

| First microRNA | Concentration threshold (range) (copies/μl) |
|---|---|
| miR-223 | 550.6 (226.4-804.8) |
| miR-25 | 8.005 (0.465-16.42) |
| miR-93 | 276.7 (241.8-305.8) |

If the expression level of the first microRNA is less than the concentration threshold, the step S34 is proceeded to. In the step S34, the risk of having colorectal cancer is assessed based on the ratio between the expression levels of the first microRNA and the second microRNA. For example, a quantitative polymerase chain reaction is performed with the primers corresponding to miR-223, miR-25, and miR-93 in the step S10. If the concentration of miR-223 is 34 copies/μl, the concentration of miR-25 is 6 copies/μl, and the concentration of miR-93 is 5 copies/μl, which are all less than their corresponding concentration thresholds, the assessment step of S34 is further proceeded to. The ratios between the expression levels of miR-223, miR-25, and miR-93 and the expression levels of miR-221, miR-222, miR-21, miR-93, miR-141, miR-200c, miR-191, miR-17, miR-148a, miR-106a, miR-195, miR-20a, miR-181b, miR-145, miR-155, miR-106b, miR-24, miR-19b, miR-130b, and miR-18a (the second microRNAs) are respectively calculated and then compared with the detection thresholds shown in Table 2 to assess the risk of having colorectal cancer. The details of the step S34 can refer to the step S20 of the first embodiment, and are therefore omitted here.

In addition, miR-223, miR-25, and miR-93 is used as the first microRNA group in this embodiment. The expression levels of miR-223, miR-25, and miR-93 is up-regulated, namely the expression levels of the first microRNAs in the stool samples of colorectal cancer patients are greater than those of normal individuals, so they can be standards for assessing the risk of having colorectal cancer. Therefore, in the embodiment, in the step S30, a first assessment is performed and the assessing result is high risk if the expression level of the first microRNA is greater than or equal to a concentration threshold (namely “positive” in the medical inspection field). In the step S34, a second assessment is then performed on the person whose expression level of the first microRNA is less than the concentration threshold (namely “negative” in the medical inspection field) by calculating ratios and the corresponding assessment method, so possible “false negative” caused by using only the first microRNAs for assessment can be avoided. Here, “false negative” means that a patient has colorectal cancer but the cancer is not detected, and medical inspection units try to avoid this error.

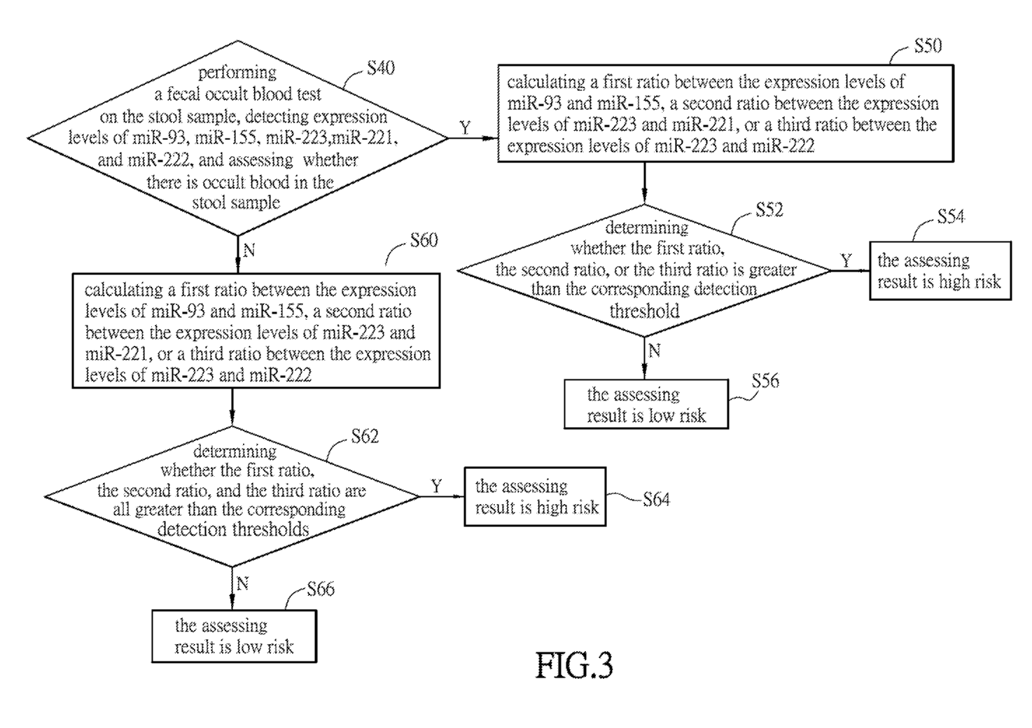

Referring to FIG. 3, it is a flow chart of the assessment method according to the third embodiment of the invention. In one embodiment, the assessment method can further be used with a fecal occult blood test (FOBT), and the specific steps are as follows: performing a fecal occult blood test on the stool sample, detecting expression levels of miR-93, miR-155, miR-223, miR-221, and miR-222, and assessing whether there is occult blood in the stool sample (step S40).

If the assessment result of the step S40 is “yes”, the stool sample having occult blood is selected, and a first ratio between the expression levels of miR-93 and miR-155, a second ratio between the expression levels of miR-223 and miR-221, or a third ratio between the expression levels of miR-223 and miR-222 is further calculated (step S50). Then, whether the first ratio, the second ratio, or the third ratio is greater than the corresponding detection threshold is determined (step S52). If the result of the step S52 is “yes”, namely if the first ratio, the second ratio, or the third ratio is greater than its corresponding detection threshold, the assessing result is high risk (step S54); otherwise the assessing result is low risk (step S56).

Moreover, if the assessment result of the step S40 is “no”, the stool sample having no occult blood is selected, and similarly a first ratio between the expression levels of miR-93 and miR-155, a second ratio between the expression levels of miR-223 and miR-221, or a third ratio between the expression levels of miR-223 and miR-222 is further calculated (step S60). Then, whether the first ratio, the second ratio, and the third ratio are greater than the corresponding detection thresholds is determined (step S62). If the result of the step S62 is “yes”, namely if the first ratio, the second ratio, and the third ratio are all greater than the corresponding detection thresholds, the assessing result is high risk (step S64); otherwise the assessing result is low risk (step S66).

As mentioned above, a fecal occult blood test is performed on the stool sample and the expression levels of miR-93, miR-155, miR-223, miR-221, and miR-222 are detected in the step S40 of the assessment method of the third embodiment. However, the fecal occult blood test and the detection of expression levels can be performed simultaneously or separately, and the invention is not limited thereto. Here, the method for detecting the expression levels of miR-93, miR-155, miR-223, miR-221, and miR-222 can refer to the step S10 of the first embodiment, so it is not repeated here. Moreover, in the step S40, whether the stool sample has occult blood is assessed based on the result of the fecal occult blood test which is performed on the stool sample. Then, that which has occult blood (namely “positive” generally called in the medical inspection field) is selected to proceed to the steps S50-S56, and that which has no occult blood (namely “negative” generally called in the medical inspection field) is selected to proceed to the steps S60-S66. In other words, the assessment processes of the steps S50-S56 and the steps S60-S66 are substantially the same, but only the assessment objects (the stool samples have occult blood or have no occult blood) and the assessment standards are slightly different. Therefore, the steps S50-S56 are illustrated for example below.

In the step S50, the first ratio between the expression levels of miR-93 and miR-155, the second ratio between the expression levels of miR-223 and miR-221, or the third ratio between the expression levels of miR-223 and miR-222 is calculated first. In the embodiment, the specific value of miR-155/miR-93 (the first ratio), the specific value of miR-223/miR-221 (the second ratio), and the specific value of miR-223/miR-222 (the third ratio) are calculated based on the combinations of the first microRNA and the second microRNA shown in Table 2 and the expression levels of miR-93, miR-155, miR-223, miR-221, and miR-222 obtained in the step S40. Subsequently, in the step S52, the first ratio, the second ratio or the third ratio obtained above are compared with the detection thresholds shown in Table 2 to determine whether they are greater than the corresponding detection thresholds. In detail, it is to determine whether the first ratio is greater than 0.0051, whether the second ratio is greater than 9.563, and whether the third ratio is greater than 8.846. If one of them is greater than the corresponding detection threshold, the assessing result can be high risk (step S54). Otherwise, if the first ratio, the second ratio, and the third ratio are all less than the corresponding detection thresholds, the assessing result can be low risk (step S56).

In other words, the steps S50-S56 are to select the stool sample having occult blood and assess the risk of having colorectal cancer for the individual based on the first ratio between the expression levels of miR-93 and miR-155, the second ratio between the expression levels of miR-223 and miR-221, or the third ratio between the expression levels of miR-223 and miR-222. If the first ratio, the second ratio, or the third ratio is greater than its corresponding detection threshold, the assessing result is high risk. Correspondingly, the steps S60-S66 are to select the stool sample having no occult blood and assess the risk of having colorectal cancer for the individual based on the first ratio between the expression levels of miR-93 and miR-155, the second ratio between the expression levels of miR-223 and miR-221, and the third ratio between the expression levels of miR-223 and miR-222. If the first ratio, the second ratio, and the third ratio are all greater than respectively corresponding detection thresholds, the assessing result is high risk; otherwise, if the first ratio, the second ratio, or the third ratio is less than its corresponding detection threshold, the assessing result is low risk.

Therefore, in the third embodiment, the second assessment is further performed on that which is assessed to have occult blood (positive) based on the preliminary result of the fecal occult blood test for the stool sample by using miR-155/miR-93 (the first ratio), miR-223/miR-221 (the second ratio), and miR-223/miR-222 (the third ratio) so as to further determine that the stool sample with occult blood (positive) simultaneously has high risk of having colorectal cancer and exclude the false positive at the same time. In detail, the fecal occult blood test for the stool sample often has the detection result of “false positive” caused by menstruation, hemorrhoids or constipation bleeding, hematuria, or other reasons. In the assessment method of the third embodiment, the second assessment is subsequently performed by using three ratios of microRNAs, so it can further exclude results of false positives and have relatively accurate results.

Moreover, in the third embodiment, the second assessment is similarly further performed on that which has no occult blood (negative) by using miR-155/miR-93 (the first ratio), miR-223/miR-221 (the second ratio), and miR-223/miR-222 (the third ratio), namely the steps S62-S66, so as to further determine that the stool sample with no occult blood (negative) has high risk of having colorectal cancer, namely to find out a false negative.

Preferably, in detail, whether the stool sample has occult blood cannot be assessed by a fecal occult blood test because the colorectal cancer patient has no bloody stools, and then a false negative occurs. The second assessment is subsequently performed by using three ratios of microRNAs, so it can further find out results of false negatives and have relatively accurate results.

In addition, the fourth embodiment also provides a marker for assessing the risk of having colorectal cancer for an individual by a stool sample obtained from the individual. The marker includes the first microRNA and the second microRNA. There is a statistically significant difference between a ratio between the expression levels of the first microRNA and the second microRNA in the stool sample obtained from a colorectal cancer patient and the ratio in a control stool sample. The first microRNA group and the second microRNA group in this embodiment are the same as the first embodiment as shown in Table 1.

The marker in the fourth embodiment is used for assessing the risk of having colorectal cancer for an individual, and its steps and effect are the same as the first embodiment. The stool samples are collected from colorectal cancer patients and healthy individuals, and the ratios between the expression levels of the first microRNAs and the second microRNAs shown in Table 1 in the stool samples are detected. The detailed steps can refer to the first embodiment, so they are not repeated here. Moreover, the detection result shows that a colorectal cancer patient has at least one ratio between the expression levels of the first microRNA and the second microRNA in the stool sample statistically significantly different from the ratio of a healthy individual. That is to say, the term “control stool sample” recited in this embodiment means the stool sample of the healthy individual used as a comparison basis for colorectal cancer patient.

As mentioned above, the assessment method and the markers are used to assess the risk of having colorectal cancer for an individual based on the ratio between the expression levels of the first microRNA and the second microRNA. Thus, they provide the assessment result of the non-invasive detection with high accuracy.

This assessment method collected stool samples from 144colorectal cancer patients and 390 healthy individuals and analyzed the expression levels of various types of microRNAs to find out markers (the microRNAs listed in Table 1) which are also known as biomarkers for assessing the risk of having colorectal cancer and the detection thresholds (as shown in Table 2) corresponding to the markers. In other words, the assessment method is established by the markers and the corresponding detection thresholds. In the below descriptions, Experimental example 1 illustrates that the assessment method is adapted to assess the risk of having colorectal cancer for an individual, and the following experimental examples illustrate that the assessment method has preferable assessment results.

### EXPERIMENTAL EXAMPLE 1

### The Assessment Method may be used for Assessing the Risk of Having Colorectal Cancer for an Individual

Assessment Objects and Stool Samples

In this experimental example, stool samples of 144 colorectal cancer (CRC) patients and 390 healthy individuals were collected from Chang Gung Memorial Hospital in Taiwan, and the expression level of each microRNA listed in Table 1 is analyzed. Cancer is staged according to the 2009 American Joint Committee on Cancer staging criteria (7th edition), and clinicopathological factors are recorded simultaneously, including age, sex, and immunological fecal occult blood test (iFOBT) data. For sample collection, CRC patients donate their stool residuum samples which are leftovers of routine iFOBT before any kind of treatment. For the healthy control group, stool samples were obtained from a Healthy-Check Center in Taoyuan Chang Gung Memorial Hospital. Participants undergo colonoscopy and all have negative findings defined by absence of neoplasia, benign polyps with pathologically approved hyperplasia, or <10 mm tubular adenoma, and their iFOBT data were also collected. All patients and healthy individuals were provided with written informed consent, and the study was approved by the institutional review board of Chang Gung Memorial Hospital.

In the experimental example, the collection container and the kit for iFOBT are used, for example OC-Sensor Diana Latex Reagent (Eiken Chemical, Tokyo, Japan) kit. In detail, the stool sample is dipped with a swab or a stool collection device and then the swab or the device is put into a preservation buffer, and the stool sample attached to the swab or the stool collection device is evenly dissolved in the preservation buffer by thorough vortex. After thorough vortex, the preservation buffer having the stool sample on the swab or the device is squeeze out into a microcentrifuge tube (Eppendorf) or other containers for subsequent experiments. The treated stool sample is stored at −80° C. until the subsequent experiments are performed.

Extraction of microRNA

In this experimental example, total RNA including microRNA is extracted from the stool sample by miRNeasy Mini Kit (QIAGEN, CA, USA). First, debris is removed from the treated stool sample mentioned above by high speed centrifugation, and 300 μL of supernatant of the treated stool sample is then collected in the collection tube of miRNeasy Mini Kit. Then, buffers are added according to the manufacturer's instructions of miRNeasy Mini Kit. Last, it is eluted with 30 μL of RNase-free water to obtain about 30 μL of solution of total RNA and microRNA, and the solution may be stored at −80° C. until use.

Reverse Transcription Polymerase Chain Reaction (RT-PCR)

Subsequently, the reverse transcription polymerase chain reaction (RT-PCR) is performed using TaqMan miRNA Reverse Transcription Kit (Applied Biosystems, Foster City, Calif.) in this experimental example. The above extracted total RNA and microRNAs act as templates, and complementary DNAs (cDNAs) are formed by reverse transcription. In the experimental example, primers used in RT-PCR and primers and probes used in subsequent Quantitative-PCR are purchased by entering the corresponding accession numbers (Accession No.) on the website of Applied Biosystems (http://bioinfo.appliedbiosystems.com/genome-database/mirna.html). The corresponding accession numbers of the primers used in RT-PCR and the primers and probes used in Quantitative-PCR for the first microRNA and the second microRNA used in the experimental example are shown in following Table 4.

TABLE 4
Corresponding accession numbers of the reverse transcription primers
used in RT-PCR and the PCR primers and probes used in Quantitative-
PCR for the microRNAs used in the experimental example

| microRNA | Accession No. |
|---|---|
| miR-223 | MI0000300 |
| miR-25 | MI0000082 |
| miR-93 | MI0000095 |
| miR-221 | MI0000298 |
| miR-222 | MI0000299 |
| miR-21 | MI0000077 |
| miR-141 | MI0000457 |
| miR-200c | MI0000650 |
| miR-191 | MI0000465 |
| miR-17 | MI0000071 |
| miR-148a | MI0000253 |
| miR-106a | MI0000113 |
| miR-195 | MI0000489 |
| miR-20a | MI0000076 |
| miR-181b | MI0000270 |
| miR-145 | MI0000461 |
| miR-155 | MI0000681 |
| miR-106b | MI0000734 |
| miR-24 | MI0000080 |
| miR-19b | MI0000074 |
| miR-130b | MI0000748 |
| miR-18a | MI0000072 |

Then, according to the manufacturer's instructions of TaqMan miRNA Reverse Transcription Kit, the primers purchased by the above method, the total RNA, and the microRNAs (templates) are mixed with other reaction reagents to perform reverse transcription reaction. Here, the thermal-cycling conditions are performed as follows: 16° C. for 30 minutes, followed by 50 cycles at 20° C. for 30 seconds, 42° C. for 30 seconds, 50° C. for 1 second, and finally 70° C. for 10 minutes. Then, cDNAs are obtained.

Quantitative Polymerase Chain Reaction (Quantitative-PCR)

In this experimental example, the quantitative polymerase chain reaction (Quantitative-PCR, qPCR) is performed with TaqMan Human MiRNA Assay (Applied Biosystems, Foster City, Calif.). First, the obtained cDNAs act as templates for qPCR, and the corresponding primers and probes are added which are purchased on the website of Applied Biosystems according to the accession numbers shown in Table 4. All required parameters of qPCR are set according to TaqMan® MicroRNA Assays Protocol (2006 edition, Part Number 4364031, Rev. B) to detect the corresponding first microRNA and second microRNA, and then the concentration (copies/μl) or the Cq value of the corresponding microRNA in the stool sample of the assessment object is obtained.

TABLE 5
Expression levels of the first microRNA and the second microRNA
of assessment objects in Experimental example 1

| | | | First microRNA/ | |
|---|---|---|---|---|
| Sample | Concentration (copies/μl) | | second microRNA | |
| No. | miR-223 | miR-221 | miR-223/miR-221 | Assessment |
| N-1 | 34.1 | 25.2 | 1.357 | Low risk |
| N-2 | 279.2 | 236.4 | 1.181 | Low risk |
| N-3 | 5.9 | 50.7 | 0.117 | Low risk |
| N-4 | 275.7 | 170.9 | 1.613 | Low risk |
| N-5 | 189.7 | 65.1 | 2.914 | Low risk |
| N-6 | 209.8 | 184.4 | 1.138 | Low risk |
| CRC-1 | 956.0 | 38.1 | 25.124 | High risk |
| CRC-2 | 6817.0 | 342.0 | 19.932 | High risk |
| CRC-3 | 2109.8 | 14.1 | 149.915 | High risk |
| CRC-4 | 4528.8 | 39.9 | 113.457 | High risk |
| CRC-5 | 1123.6 | 21.3 | 52.820 | High risk |
| CRC-6 | 18253.9 | 84.9 | 215.120 | High risk |

Table 5 shows the detection results of the stool samples of 6 patients with diagnosed colorectal cancer and 6 healthy individuals collected in the experimental example. Here, in the column “Sample No.”, “N” indicates the normal group, namely the detection results of the stool samples of the healthy individuals, and “CRC” indicates the colorectal cancer group, namely the detection results of the stool samples of the patients with diagnosed colorectal cancer. 6 healthy individuals and 6 patients with diagnosed colorectal cancer are respectively taken as the normal group and the colorectal cancer group, and they are numbered by 1-6. In the experimental example, after the concentrations of the first microRNAs (miR-223) and the second microRNA (miR-221) in the stool samples of the assessment objects (N1-N6 and CRC1-CRC6) are respectively obtained according to the steps mentioned above, the risk of having colorectal cancer for an individual can be assessed based on the ratio between the expression levels of the first microRNA and the second microRNA.

As shown in Table 2, when the first microRNA is miR-223 and the second microRNA is miR-221, the detection threshold is 9.563. Therefore, if the specific value of miR-223/miR-221 is greater than 9.563, it may be assessed to be high risk and referred to a positive in general clinical detection. If the specific value of miR-223/miR-221 is less than 9.563, it may be assessed to be low risk and referred to a negative in general clinical detection. As shown in Table 4, in the normal group (N1-N6), the specific values of miR-223/miR-221 are all less than 3, and thus they are assessed to be low risk. Further, in the colorectal cancer group (CRC1˜CRC6), the specific values of miR-223/miR-221 are all greater than 19, and thus they may be assessed to be high risk. Therefore, the results shown in Experimental example 1 can verify that this assessment method can be used for assessing the risk of having colorectal cancer for an individual indeed.

### EXPERIMENTAL EXAMPLE 2

### The Comparison of Effects between the Assessment Method and the Immunological Fecal Occult Blood Test

In Experimental example 2, the concentrations (expression levels) of the first microRNAs and the second microRNAs listed in Table 1 in the stool samples are detected according to the collection method and the method for detecting the expression level of Experimental example 1, and the stool samples are collected from 390 healthy individuals and 144 patients with diagnosed colorectal cancer in Experimental example 1.

Subsequently, receiver operating characteristic curves (ROC curves) are plotted with PASW Statistics 18.0 using the ratios between the first microRNAs and the second microRNAs in different combinations according to Table 2 and the source of each stool sample which is from an individual in the healthy control group or a colorectal cancer patient. Then, the area under the ROC curve (AUC) is calculated to obtain corresponding Youden Index acting as the detection threshold, and the cut-off point represents that the sum of its specificity and sensitivity is the maximum. The area under the ROC curve (AUC) may be used for evaluating the probability of correct identification of the assessment method used, thus determining the validity of the detection, also known as diagnostic accuracy. It is hereinafter referred to the AUC value. The confidence interval of Experimental example 2 is stated at the 95% confidence level, and it is statistically significant that the obtained p-value is less than 0.05.

Similarly, by the same data processing method, receiver operating characteristic curves (ROC curves) are calculated with PASW Statistics 18.0 directly using the concentrations of the first microRNAs and the second microRNAs, and the corresponding AUC values and their p-values are obtained to evaluate the validity of the assessment method by comparing the AUC values. In this experimental example and the following experimental examples, all p-values of AUC values are less than 0.05 except those specially mentioned.

Additionally, the multipliers between the ratios between the expression levels of each first microRNA and second microRNA shown in Table 2 in the stool samples of colorectal cancer patients and those of healthy normal group are calculated in the experimental example, and then the Mann-Whitney U test is performed on the obtained multipliers. If the obtained p-value is less than 0.05, it is defined to be statistically significant.

In the experimental example, the detection thresholds shown in Table 2 act as standards. It is determined to be positive (P) when the ratio between the expression levels of the first microRNA and the second microRNA is greater than the detection threshold, and it is determined to be negative (N) when that is less than the maximum of the detection threshold. For example, it is determined to be positive when the specific value of miR-223/miR-221 is greater than 9.563, and it is determined to be negative when that is less than 9.563. Then, in the stool samples determined to be positive, if the stool sample is among the samples obtained from “144 patients with diagnosed colorectal cancer”, it is a true positive (TP), and if the stool sample is among the samples obtained from “390 healthy individuals”, it is a false positive (FP). Similarly, in the stool samples determined to be negative, if such stool sample is among the samples obtained from “390 healthy individuals”, it is a true negative (TN), and if such stool sample is among the samples obtained from “144 patients with diagnosed colorectal cancer”, it is a false negative (FN). The numbers of above “true positives (TP)”, “false positives (FP)”, “true negatives (TN)”, and “false negatives (FN)” are calculated to obtain sensitivity and specificity of each combination of the first microRNA and the second microRNA shown in Table 6. Here, sensitivity is “TP/(TP+FP)”, namely true positives (TP), which are diagnosed to have colorectal cancer, over the samples determined to be positive (P), and specificity is “TN/(TN+FN)”, namely true negatives (TN), which are from healthy individual samples, over the samples determined to be negative (N).

The AUC values, the detection thresholds, the sensitivities, and the specificities of using the ratios between the expression levels of the first microRNA and the second microRNA shown in Table 2 for assessment according to the above experimental methods of this experimental example are shown below. The AUC values listed in Table 6 are all statistically significant (p<0.05). Simultaneously, in Table 6, p-values of the ratios of the ratios between the expression levels of the first microRNA and the second microRNA in the stool samples of colorectal cancer patients to those of healthy normal group (the multipliers of cancer patients/healthy individuals) are all less than 0.05. Therefore, in Table 6, the differences between the ratios between the expression levels of the first microRNA and the second microRNA in the stool samples of colorectal cancer patients and those of healthy normal group are statistically significant.

TABLE 6
Detection thresholds (range), specificities, and sensitivities of using the ratios between expression
levels of the first microRNA and the second microRNA shown in Table 2 for assessment

| | | | | | Multiplier of |
|---|---|---|---|---|---|
| | | Detection | Sensitivity | Specificity | cancer |
| | AUC | threshold | (95% confidence | (95% confidence | patients/healthy |
| | value | (range) | interval) | interval) | individuals |
| miR-223/miR-221 | 0.808 | 9.563 | 73.61% | 81.28% | 7.43 |
| | | (4.143-13.92) | (65.62%-80.60%) | (77.05%-85.03%) | |
| miR-223/miR-222 | 0.801 | 8.846 | 68.75% | 81.28% | 8.55 |
| | | (4.798-12.54) | (60.50%-76.21%) | (77.05%-85.03%) | |
| miR-223/miR-21 | 0.794 | 0.6263 | 81.94% | 64.36% | 9.48 |
| | | (0.317-0.787) | (74.67%-87.85%) | (59.38%-69.12%) | |
| miR-223/miR-93 | 0.788 | 1.239 | 65.97% | 82.82% | 8.18 |
| | | (0.676-1.897) | (57.62%-73.65%) | (78.70%-86.43%) | |
| miR-223/miR-141 | 0.785 | 20.33 | 72.22% | 73.33% | 8.19 |
| | | (12.66-28.45) | (64.15%-79.36%) | (68.65%-77.66%) | |
| miR-223/miR-200c | 0.772 | 3.706 | 81.25% | 64.1% | 9.18 |
| | | (1.697-5.152) | (73.90%-87.26%) | (59.12%-68.87%) | |
| miR-223/miR-191 | 0.772 | 42.41 | 80.56% | 62.05% | 6.84 |
| | | (25.11-58.04) | (73.14%-86.67%) | (57.03%-66.89%) | |
| miR-223/miR-17 | 0.764 | 2.838 | 75.69% | 66.15% | 5.81 |
| | | (1.509-3.541) | (67.85%-82.45%) | (61.22%-70.84%) | |
| miR-223/miR-148a | 0.754 | 137.5 | 63.19% | 78.21% | 5.96 |
| | | (64.19-205.4) | (54.76%-71.07%) | (73.77%-82.20%) | |
| miR-223/miR-106a | 0.749 | 9.550 | 58.33% | 82.31% | 6.16 |
| | | (5.79-13.95) | (49.83%-66.48%) | (78.15%-85.97%) | |
| miR-223/miR-195 | 0.746 | 46.19 | 52.78% | 83.33% | 4.49 |
| | | (29.43-67.23) | (44.29%-61.15%) | (79.26%-86.90%) | |
| miR-223/miR-20a | 0.745 | 9.458 | 72.92% | 69.23% | 5.66 |
| | | (5.79-12.94) | (64.89%-79.98%) | (64.39%-73.78%) | |
| miR-223/miR-181b | 0.742 | 61.39 | 70.83% | 68.97% | 5.79 |
| | | (24.5-89.83) | (62.68%-78.10%) | (64.12%-73.53%) | |
| miR-223/miR-145 | 0.726 | 96.44 | 53.47% | 82.31% | 18.01 |
| | | (34.58-168.9) | (44.98%-61.82%) | (78.15%-85.97%) | |
| miR-223/miR-155 | 0.720 | 128.4 | 61.11% | 75.13% | 6.51 |
| | | (63.18-200) | (52.64%-69.12%) | (70.53%-79.34%) | |
| miR-223/miR-106b | 0.720 | 4.233 | 63.89% | 73.85% | 5.89 |
| | | (1.689-5.794) | (55.47%-71.72%) | (69.19%-78.14%) | |
| miR-223/miR-24 | 0.714 | 47.21 | 56.94% | 74.1% | 5.51 |
| | | (23.65-63.29) | (48.44%-65.16%) | (69.45%-78.38%) | |
| miR-223/miR-19b | 0.685 | 41.34 | 64.58% | 65.38% | 4.84 |
| | | (19.41-56.91) | (56.19%-72.37%) | (60.43%-70.10%) | |
| miR-223/miR-130b | 0.676 | 13.93 | 72.92% | 55.9% | 6.41 |
| | | (4.649-26.92) | (64.89%-79.98%) | (50.81%-60.89%) | |
| miR-223/miR-18a | 0.698 | 42.98 | 57.64% | 73.33% | 5.03 |
| | | (19.5-66.67) | (49.13%-65.82%) | (68.65%-77.66%) | |
| miR-25/miR-221 | 0.702 | 0.4366 | 54.17% | 78.97% | 5.40 |
| | | (0.21-0.666) | (45.67%-62.49%) | (74.59%-82.91%) | |
| miR-25/miR-222 | 0.719 | 0.2664 | 59.72% | 77.95% | 4.77 |
| | | (0.139-0.386) | (51.23%-67.80%) | (73.50%-81.97%) | |
| miR-25/miR-21 | 0.718 | 0.05433 | 56.25% | 78.97% | 5.76 |
| | | (0.027-0.076) | (47.74%-64.49%) | (74.59%-82.91%) | |
| miR-25/miR-93 | 0.690 | 0.05303 | 54.17% | 79.23% | 10.77 |
| | | (0.017-0.092) | (45.67%-62.49%) | (74.86%-83.15%) | |
| miR-25/miR-141 | 0.708 | 1.061 | 50.69% | 83.33% | 3.83 |
| | | (0.714-1.897) | (42.24%-59.12%) | (79.26%-86.90%) | |
| miR-25/miR-200c | 0.692 | 0.2325 | 59.72% | 71.03% | 5.44 |
| | | (0.132-0.356) | (51.23%-67.80%) | (66.25%-75.48%) | |
| miR-25/miR-191 | 0.688 | 1.118 | 63.19% | 70.77% | 3.44 |
| | | (0.965-2.577) | (54.76%-71.07%) | (65.98%-75.24%) | |
| miR-25/miR-17 | 0.657 | 0.2446 | 45.83% | 82.31% | 3.27 |
| | | (0.125-0.363) | (37.51%-54.33%) | (78.15%-85.97%) | |
| miR-25/miR-148a | 0.665 | 8.776 | 46.53% | 84.62% | 5.14 |
| | | (3.382-14.08) | (38.18%-55.02%) | (80.65%-88.05%) | |
| miR-25/miR-106a | 0.651 | 0.1484 | 65.28% | 66.67% | 4.04 |
| | | (0.061-0.202) | (56.90%-73.01%) | (61.75%-71.33%) | |
| miR-25/miR-195 | 0.653 | 0.4772 | 74.31% | 52.82% | 2.47 |
| | | (0.292-0.724) | (66.37%-81.22%) | (47.73%-57.87%) | |
| miR-25/miR-20a | 0.646 | 0.2475 | 65.97% | 61.28% | 3.42 |
| | | (0.125-0.36) | (57.62%-73.65%) | (56.25%-66.14%) | |
| miR-25/miR-181b | 0.657 | 1.766 | 56.25% | 73.08% | 3.16 |
| | | (0.998-3.883) | (47.74%-64.49%) | (68.38%-77.42%) | |
| miR-25/miR-145 | 0.648 | 1.176 | 52.08% | 73.59% | 3.78 |
| | | (0.972-2.98) | (43.61%-60.47%) | (68.92%-77.90%) | |
| miR-25/miR-155 | 0.635 | 1.557 | 61.11% | 66.67% | 3.57 |
| | | (0.995-3.246) | (52.64%-69.12%) | (61.75%-71.33%) | |
| miR-25/miR-106b | 0.641 | 0.1166 | 57.64% | 67.95% | 3.83 |
| | | (0.052-0.185) | (49.13%-65.82%) | (63.07%-72.56%) | |
| miR-25/miR-24 | 0.649 | 1.143 | 44.44% | 81.79% | 1.00 |
| | | (0.996-2.376) | (36.17%-52.95%) | (77.60%-85.50%) | |
| miR-25/miR-19b | 0.626 | 1.105 | 53.47% | 75.38% | 1.33 |
| | | (0.993-1.873) | (44.98%-61.82%) | (70.80%-79.58%) | |
| miR-25/miR-130b | 0.629 | 1.008 | 47.92% | 74.62% | 1.46 |
| | | (0.996-1.988) | (39.53%-56.39%) | (69.99%-78.86%) | |
| miR-25/miR-18a | 0.628 | 1.041 | 52.08% | 74.87% | 1.97 |
| | | (1-1.96) | (43.61%-60.47%) | (70.26%-79.10%) | |
| miR-17/miR-93 | 0.613 | 0.1135 | 88.19% | 30.51% | 1.42 |
| | | (0.084-0.151) | (81.77%-92.97%) | (25.98%-35.35%) | |
| miR-106a/miR-93 | 0.630 | 0.1522 | 69.44% | 54.36% | 1.68 |
| | | (0.104-0.191) | (61.23%-76.84%) | (49.27%-59.38%) | |
| miR-195 miR-93 | 0.607 | 0.1517 | 29.86% | 91.03% | 1.83 |
| | | (0.089-0.209) | (22.53%-38.04%) | (87.74%-93.67%) | |
| miR-20a/miR-93 | 0.603 | 0.2473 | 36.81% | 83.33% | 1.68 |
| | | (0.172-0.289) | (28.93%-45.24%) | (79.26%-86.90%) | |
| miR-181b/miR-93 | 0.579 | 0.01116 | 64.58% | 50.51% | 1.52 |
| | | (0.007-0.014) | (56.19%-72.37%) | (45.44%-55.58%) | |
| miR-155/miR-93 | 0.577 | 0.005128 | 74.31% | 45.13% | 1.33 |
| | | (0.004-0.006) | (66.37%-81.22%) | (40.11%-50.22%) | |
| miR-24/miR-93 | 0.560 | 0.1271 | 40.28% | 76.41% | 1.83 |
| | | (0.038-0.187) | (32.20%-48.77%) | (71.88%-80.54%) | |
| miR-19b/miR-93 | 0.607 | 0.04282 | 55.56% | 64.1% | 3.54 |
| | | (0.012-0.06) | (47.05%-63.83%) | (59.12%-68.87%) | |
| miR-18a/miR-93 | 0.592 | 0.03339 | 61.11% | 55.64% | 1.69 |
| | | (0.018-0.043) | (52.64%-69.12%) | (50.55%-60.64%) | |

In addition, immunological fecal occult blood tests (iFOBT) are performed on the stool samples collected in Experimental example 1, and the sensitivity and the specificity of iFOBT are found to be 55.2% and 66.2% respectively. Therefore, as shown in the above table, whether the ratio of the combination of miR-223 and miR-221, miR-223 and miR-222, miR-223 and miR-93, miR-223 and miR-141, miR-223 and miR-148a, miR-223 and miR-106a, miR-223 and miR-20a, miR-223 and miR-181b, miR-223 and miR-155, miR-223 and miR-106b, miR-223 and miR-24, miR-223 and miR-18a, miR-25 and miR-222, miR-25 and miR-21, miR-25 and miR-200c, miR-25 and miR-191, miR-25 and miR-106a, miR-25 and miR-181b, miR-25 and miR-155, or miR-25 and miR-106b is used for assessing the risk of having colorectal cancer for an individual, they all have relatively high sensitivity and specificity in comparison with iFOBT. Therefore, the assessment method of the first embodiment can more effectively assess the risk of having colorectal cancer for an individual in comparison with the iFOBT method.

### EXPERIMENTAL EXAMPLE 3

### The Comparison of Effects between the Assessment Method and Using a Single microRNA for Assessment

The experimental process and data calculation of Experimental example 3 may both refer to Experimental example 2 mentioned above. In this experimental example, compared with using an expression level of a single microRNA (first microRNA or second microRNA) for assessment, using the ratio between expression levels of the first microRNA and the second microRNA shown in Table 2 has better result. For example, the obtained AUC values (diagnostic accuracy) of miR-223/miR-221, miR-223/miR-222, miR-223/miR-21, miR-223/miR-93, miR-25/miR-221, miR-25/miR-222, miR-25/miR-21, miR-25/miR-93, miR-25/miR-141, miR-25/miR-200c, and miR-25/miR-191 are greater than the AUC values (diagnostic accuracy) of using the first microRNA (miR-223 or miR-25) only or using the second microRNA (miR-221, miR-222, miR-21, miR-93, miR-141, miR-200c, or miR-191) only. It shows that using the ratios between the expression levels of the first microRNAs and the second microRNAs mentioned above for assessing the risk of having colorectal cancer for an individual is relatively effective in comparison with using the corresponding first microRNA (miR-223 or miR-25) only or using the corresponding second microRNA (miR-221, miR-222, miR-21, miR-93, miR-141, miR-200c, or miR-191) only.

TABLE 7
AUC values for ratios of combinations of the
first microRNA and the second microRNA of the
assessment objects in Experimental example 3

| | | | | AUC of |
|---|---|---|---|---|
| | | | | first |
| | | AUC of | AUC of | microRNA/ |
| First | Second | first | second | second |
| microRNA | microRNA | microRNA | microRNA | microRNA |
| miR-223 | miR-221 | 0.785 | 0.564 | 0.808 |
| | miR-222 | | 0.552 | 0.801 |
| | miR-21 | | 0.520 | 0.794 |
| | miR-93 | | 0.558 | 0.788 |
| miR-25 | miR-221 | 0.674 | 0.564 | 0.702 |
| | miR-222 | | 0.552 | 0.719 |
| | miR-21 | | 0.520 | 0.718 |
| | miR-93 | | 0.558 | 0.690 |
| | miR-141 | | 0.500 | 0.708 |
| | miR-200c | | 0.506 | 0.692 |
| | miR-191 | | 0.565 | 0.688 |

### EXPERIMENTAL EXAMPLE 4

### The Comparison of Expression Variation of the First microRNAs and the Second microRNAs Shown in Table 1 Between Different Samples

Experimental example 4 is to compare variation levels of the first microRNAs and the second microRNAs shown in Table 1 in stool samples, tissue samples and blood samples respectively. Experimental example 4 is the same as Experimental example 1. The stool samples of 144 patients with diagnosed colorectal cancer (the colorectal cancer group) and 390 healthy individuals (the normal group) are collected for analysis. As to the tissue samples, cancerous tissue samples of 81 patients with diagnosed colorectal cancer (the colorectal cancer group) and large intestine tissue samples of non-pathological tissues of the same patients (the normal group) are collected by surgery for analysis. Regarding the blood samples, the blood samples of 215 patients with diagnosed colorectal cancer (the colorectal cancer group) and 173 healthy individuals (the normal group) are drawn for analysis. Then, extraction of microRNA, RT-PCR, and qPCR are performed on the stool samples, the tissue samples and the blood samples according to the steps of Experimental example 1 to obtain the expression levels of each first microRNA and each second microRNA.

Subsequently, specific values of expression levels of the same microRNA (for example miR-223) between the colorectal cancer group and the normal group in different samples are calculated, and the normal group is used as the denominator to obtain a multiplier in comparison with the normal group, and Mann-Whitney U test is performed simultaneously. If the obtained p-value is less than 0.05, the difference is defined to be statistically significant. Therefore, if the expression level of the colorectal cancer group is equal to that of the normal group, the specific value is 1; if the expression level of the colorectal cancer group is greater, the specific value is greater than 1; if the expression level of the normal group is greater, the specific value is between 0 and 1. Other detailed steps may refer to the above description, so they are not repeated here.

TABLE 8
Variation levels of expression of microRNAs in the stool samples,
the tissue samples and the blood samples respectively

| | Tissue sample | | Stool sample | | | |
|---|---|---|---|---|---|---|
| | | p- | | p- | Blood sample | |
| microRNA | Multiplier | value | Multiplier | value | Multiplier | p-value |
| miR-223 | 3.01 | 0.000 | 84.50 | 0.000 | 2.92 | 0.000 |
| miR-25 | 1.51 | 0.000 | 6.27 | 0.000 | 2.00 | 0.000 |
| miR-93 | 2.28 | 0.000 | 1.67 | 0.039 | 1.88 | 0.000 |
| miR-221 | 2.26 | 0.000 | 3.39 | 0.024 | 2.58 | 0.000 |
| miR-222 | 1.65 | 0.043 | 2.09 | 0.063 | 1.58 | 0.000 |
| miR-21 | 2.50 | 0.000 | 0.75 | 0.479 | 1.62 | 0.000 |
| miR-141 | 1.67 | 0.000 | 1.21 | 0.998 | 0.67 | 0.000 |
| miR-200c | 1.89 | 0.000 | 0.98 | 0.822 | 2.89 | 0.006 |
| miR-191 | 2.02 | 0.000 | 6.13 | 0.016 | 3.30 | 0.000 |
| miR-17 | 3.95 | 0.000 | 3.55 | 0.000 | 1.79 | 0.000 |
| miR-148a | 2.01 | 0.000 | 2.75 | 0.003 | 1.03 | 0.795 |
| miR-106a | 4.30 | 0.000 | 3.52 | 0.000 | 1.78 | 0.000 |
| miR-195 | 0.49 | 0.000 | 7.84 | 0.000 | 1.64 | 0.118 |
| miR-20a | 3.78 | 0.000 | 3.93 | 0.000 | 1.66 | 0.004 |
| miR-181b | 1.53 | 0.169 | 2.08 | 0.000 | 0.96 | 0.533 |
| miR-145 | 0.38 | 0.000 | 1.37 | 0.300 | 1.37 | 0.337 |
| miR-155 | 1.69 | 0.506 | 2.37 | 0.000 | 1.51 | 0.013 |
| miR-106b | 3.09 | 0.000 | 1.66 | 0.009 | 1.36 | 0.018 |
| miR-24 | 1.36 | 0.158 | 4.74 | 0.011 | 3.00 | 0.000 |
| miR-19b | 1.98 | 0.000 | 5.02 | 0.000 | 1.55 | 0.000 |
| miR-130b | 2.04 | 0.000 | 0.64 | 0.151 | 1.09 | 0.564 |
| miR-18a | 7.13 | 0.000 | 2.40 | 0.000 | 3.05 | 0.000 |

It can be seen from Table 8 that variation levels of expression of the first microRNAs and the second microRNAs shown in Table 1 are different in different kinds of samples. Moreover, even if the multiplier of variation is statistically significant in a certain kind of sample, it is not necessary that the multiplier of variation is statistically significant in the other kind of sample (e.g. miR-155, miR-181b, and miR-24). Therefore, the microRNA suitable for assessing the risk of having colorectal cancer for an individual in the tissue sample is not necessarily suitable for the stool sample.

In addition, not all variation levels of expression of microRNAs shown in Table 1 are statistically significant even in the stool samples. Accordingly, if only the variation levels of expression of microRNAs shown in Table 1 are used, not all of them can effectively assess the risk of having colorectal cancer for an individual. In contrast, as shown in Table 6, the assessment method according to the first embodiment of the invention which is using the ratio between the expression levels of the first microRNA and the second microRNA can transform the microRNAs which are not suitable for being used alone in the stool samples into effective assessment targets.

### EXPERIMENTAL EXAMPLE 5

### The Assessment Method According to the Second Embodiment can Reduce the Incorrect Assessment of False Negative

Compared with directly using the corresponding first microRNA for assessment (i.e. those do not show contents in the column of “first microRNA/second microRNA” in Table 9), the false negative can be reduced if the assessment method according to the second embodiment is used for assessing the risk of having colorectal cancer for an individual. The assessment method according to the second embodiment is using the expression level of the first microRNA for assessing first and then calculating the ratio to assess the risk of having colorectal cancer. Results are shown in Table 9.

TABLE 9
Sensitivities of the assessment method according to the second
embodiment and merely using the corresponding first microRNA

| First microRNA | First microRNA/second microRNA | Sensitivity (%) |
|---|---|---|
| miR-223 | — | 63.9 |
| miR-223 | miR-223/miR-21 | 85.4 |
| miR-223 | miR-223/miR-200c | 84.7 |
| miR-223 | miR-223/miR-191 | 84.0 |
| miR-223 | miR-223/miR-130b | 82.6 |
| miR-223 | miR-223/miR-221 | 79.9 |
| miR-223 | miR-223/miR-20a | 79.9 |
| miR-223 | miR-223/miR-19b | 79.9 |
| miR-223 | miR-223/miR-141 | 79.2 |
| miR-223 | miR-223/miR-24 | 79.2 |
| miR-223 | miR-223/miR-17 | 78.5 |
| miR-223 | miR-223/miR-222 | 77.1 |
| miR-223 | miR-223/miR-181b | 77.1 |
| miR-223 | miR-223/miR-195 | 76.4 |
| miR-223 | miR-223/miR-106a | 75.0 |
| miR-223 | miR-223/miR-148a | 72.9 |
| miR-223 | miR-223/miR-106b | 72.9 |
| miR-223 | miR-223/miR-18a | 72.9 |
| miR-223 | miR-223/miR-155 | 72.2 |
| miR-223 | miR-223/miR-145 | 71.5 |
| miR-223 | miR-223/miR-93 | 70.1 |
| miR-25 | — | 61.11 |
| miR-25 | miR-25/miR-20a | 72.92 |
| miR-25 | miR-25/miR-195 | 81.25 |
| miR-25 | miR-25/miR-106a | 70.83 |
| miR-25 | miR-25/miR-222 | 69.44 |
| miR-25 | miR-25/miR-200c | 68.06 |
| miR-25 | miR-25/miR-21 | 67.36 |
| miR-25 | miR-25/miR-106b | 67.36 |
| miR-25 | miR-25/miR-221 | 66.67 |
| miR-25 | miR-25/miR-155 | 65.97 |
| miR-25 | miR-25/miR-191 | 65.28 |
| miR-25 | miR-25/miR-24 | 65.28 |
| miR-25 | miR-25/miR-130b | 64.58 |
| miR-25 | miR-25/miR-19b | 63.89 |
| miR-25 | miR-25/miR-181b | 63.19 |
| miR-25 | miR-25/miR-145 | 63.19 |
| miR-25 | miR-25/miR-18a | 63.19 |
| miR-25 | miR-25/miR-17 | 62.50 |
| miR-25 | miR-25/miR-141 | 61.81 |
| miR-25 | miR-25/miR-93 | 61.11 |
| miR-25 | miR-25/miR-148a | 61.11 |
| miR-93 | — | 50.69 |
| miR-93 | miR-93/miR-17 | 90.97 |
| miR-93 | miR-93/miR-155 | 90.97 |
| miR-93 | miR-93/miR-106a | 77.08 |
| miR-93 | miR-93/miR-181b | 77.08 |
| miR-93 | miR-93/miR-18a | 75.69 |
| miR-93 | miR-93/miR-19b | 74.31 |
| miR-93 | miR-93/miR-20a | 65.97 |
| miR-93 | miR-93/miR-24 | 62.50 |
| miR-93 | miR-93/miR-195 | 61.11 |

It can be seen from the above table that the sensitivity of using the assessment method according to the second embodiment to assess the risk of having colorectal cancer for an individual is higher than that of directly using the corresponding first microRNA. Therefore, it can effectively reduce the false negative caused by merely using the first microRNA.

### EXPERIMENTAL EXAMPLE 6

### The Comparison between iFOBT and the Assessment Methods According to the First Embodiment and the Third Embodiment

As to collection of the stool samples, iFOBT, extraction of each microRNA, RT-PCR, qPCR, and calculation of the ratio between the expression levels of microRNAs in this experimental example, the materials and the experimental methods used are the same as Experimental example 1. In addition, because advanced polyps are quite possible to develop into colorectal cancer, the stool samples of 27 individuals with advanced polyps determined by colonoscopy are also collected and included in the analysis in this experimental example.

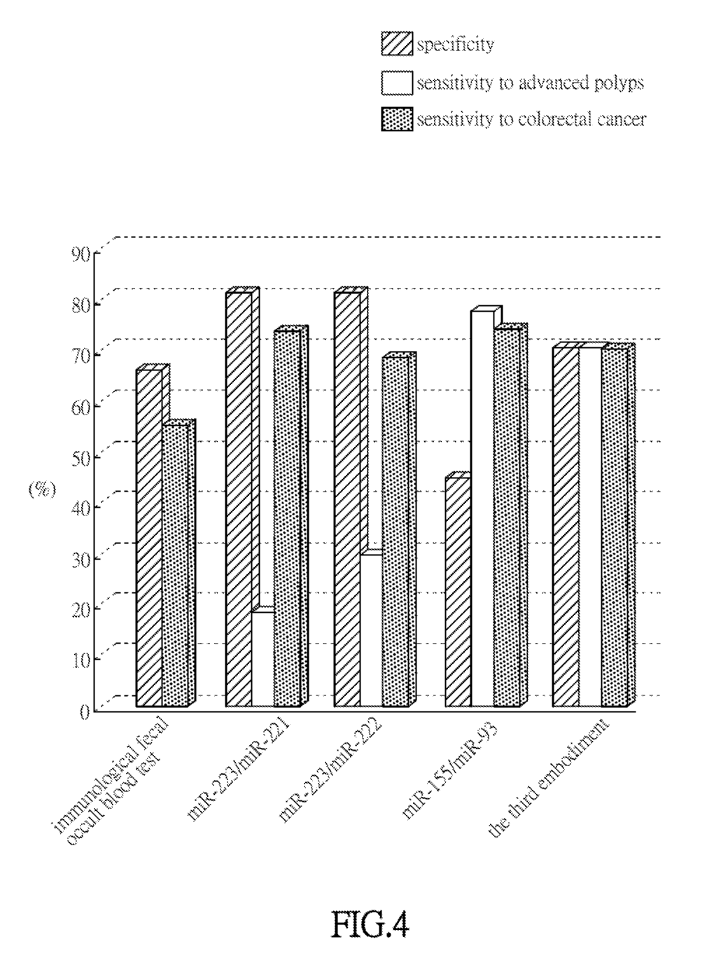

As shown in FIG. 4, it is a diagram showing the experimental result of each assessment method of Experimental example 6 of the invention. As previously described, the sensitivity and the specificity of iFOBT to colorectal cancer are 55.2% and 66.2% respectively. It can been seen from the figure that the specificity of the assessment method described in the third embodiment (the first ratio between the expression levels of miR-93 and miR-155, the second ratio between the expression levels of miR-223 and miR-221, and/or the third ratio between the expression levels of miR-223 and miR-222 are/is used to perform the assessment) is 70.51%, its sensitivity to the colorectal cancer samples is 70.14%, and its sensitivity to the advanced polyps samples is 70.37%. Therefore, assessing the risk of having colorectal cancer for an individual using the assessment method described in the third embodiment can both reduce the incorrect assessment of false positive and false negative caused by iFOBT.

Further, as to merely using a single ratio for assessment, sensitivity of using miR-223/miR-221 or miR-223/miR-222 for assessment to the advanced polyps samples is only 18.52% or 29.63% respectively, and they are both lower than that of the assessment method according to the third embodiment to the advanced polyps samples (70.37%). On the other hand, the specificity of using miR-155/miR-93 for assessment is only 45.13% which is lower than that of the assessment method according to the third embodiment (70.51%). Accordingly, by comparison, the assessment method according to the third embodiment can maintain the excellent sensitivity to the advanced polyps samples and the colorectal cancer samples while maintaining its specificity. Therefore, the assessment method according to the third embodiment can more effectively assess the risk of having colorectal cancer for an individual.

Although the present invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the present invention.

What is claimed is:

1. A method for assessing the risk of having colorectal cancer for an individual by a stool sample obtained from the individual, comprising:
detecting expression levels of a first microRNA and a second microRNA in the stool sample; and
assessing the risk of having colorectal cancer for the individual based on a ratio between the expression levels of the first microRNA and the second microRNA;
wherein the first microRNA is selected from the group consisting of miR-223, miR-25, and miR-93, the second microRNA is selected from the group consisting of miR-221, miR-222, miR-21, miR-93, miR-141, miR-200c, miR-191, miR-17, miR-148a, miR-106a, miR-195, miR-20a, miR-181b, miR-145, miR-155, miR-106b, miR-24, miR-19b, miR-130b, and miR-18a, and when the first microRNA is miR-93, the second microRNA is miR-17, miR-106a, miR-195, miR-20a, miR-181b, miR-155, miR-24, miR-19b, or miR-18a.

2. The method of claim 1, wherein the result of the step of assessing the risk of having colorectal cancer for the individual is high risk if the ratio between the expression levels of the first microRNA and the second microRNA is greater than a detection threshold.

3. The method of claim 1, wherein the first microRNA is miR-223, and the second microRNA is miR-221, miR-222, miR-21, or miR-93.

4. The method of claim 1, wherein the first microRNA is miR-25, and the second microRNA is miR-221, miR-222, miR-21, miR-93, miR-141, miR-200c, or miR-191.

5. The method of claim 1, wherein the expression level of the first microRNA is up-regulated.

6. The method of claim 1, wherein after the step of detecting expression levels of a first microRNA and a second microRNA in the stool sample, following steps are performed:
assessing the risk as high risk if the expression level of the first microRNA is greater than a concentration threshold; and
assessing the risk of having colorectal cancer for the individual based on the ratio between the expression levels of the first microRNA and the second microRNA if the expression level of the first microRNA is less than the concentration threshold, wherein the first microRNA is selected from the group consisting of miR-223, miR-25, and miR-93, the second microRNA is selected from the group consisting of miR-221, miR-222, miR-21, miR-93, miR-141, miR-200c, miR-191, miR-17, miR-148a, miR-106a, miR-195, miR-20a, miR-181b, miR-145, miR-155, miR-106b, miR-24, miR-19b, miR-130b, and miR-18a, and when the first microRNA is miR-93, the second microRNA is miR-17, miR-106a, miR-195, miR-20a, miR-181b, miR-155, miR-24, miR-19b, or miR-18a.

7. The method of claim 6, wherein the result of the step of assessing the risk of having colorectal cancer for the individual is high risk if the ratio between the expression levels of the first microRNA and the second microRNA is greater than a detection threshold.

8. The method of claim 6, wherein the expression level of the first microRNA is up-regulated.

9. A method for assessing the risk of having colorectal cancer for an individual by a stool sample obtained from the individual, comprising:
performing a fecal occult blood test on the stool sample and detecting expression levels of miR-93, miR-155, miR-223, miR-221, and miR-222;
selecting the stool sample of positive fecal occult blood test result and assessing the risk of having colorectal cancer for the individual based on a first ratio between the expression levels of miR-93 and miR-155, a second ratio between the expression levels of miR-223 and miR-221, or a third ratio between the expression levels of miR-223 and miR-222, wherein the assessing result is high risk if the first ratio, the second ratio, or the third ratio is greater than a corresponding detection threshold; and
selecting the stool sample of negative fecal occult blood test result and assessing the risk of having colorectal cancer for the individual based on the first ratio between the expression levels of miR-93 and miR-155, the second ratio between the expression levels of miR-223 and miR-221, and the third ratio between the expression levels of miR-223 and miR-222, wherein the assessing result is high risk if the first ratio, the second ratio, and the third ratio are all greater than the corresponding detection thresholds.

10. A marker for assessing the risk of having colorectal cancer for an individual in a stool sample obtained from the individual, comprising:
a first microRNA and a second microRNA, wherein the difference between a ratio between expression levels of the first microRNA and the second microRNA in at least one stool sample obtained from a colorectal cancer patient and that in a control stool sample is statistically significant;
wherein the first microRNA is selected from the group consisting of miR-223, miR-25, and miR-93, the second microRNA is selected from the group consisting of miR-221, miR-222, miR-21, miR-93, miR-141, miR-200c, miR-191, miR-17, miR-148a, miR-106a, miR-195, miR-20a, miR-181b, miR-145, miR-155, miR-106b, miR-24, miR-19b, miR-130b, and miR-18a, and when the first microRNA is miR-93, the second microRNA is miR-17, miR-106a, miR-195, miR-20a, miR-181b, miR-155, miR-24, miR-19b, or miR-18a.
